(19) United States
(12) Patent Application Publication
(10) Pub. No.: US 2026/0090852 A1
(43) Pub. Date: Apr. 2, 2026

(54) INSTRUMENT ARTICULATION DAMPENING

(71) Applicant: AURIS HEALTH, INC., Santa Clara, CA (US)

(72) Inventors: James Hayes, Redwood City, CA (US); Adrian Hairrell, San Francisco, CA (US)

(73) Assignee: AURIS HEALTH, INC., Santa Clara, CA (US)

(21) Appl. No.: 18/904,067

(22) Filed: Oct. 1, 2024

(51) Int. Cl. A61B 34/35; A61B 34/00

(52) U.S. Cl. CPC A61B 34/35; A61B 34/71; A61B 2034/715

(57) ABSTRACT

An instrument, such as a robotically-articulable medical instrument, includes a base, a shaft that projects from the base, the shaft having one or more articulation pull wires disposed at least partially therein, a robotic drive input, a pulley having at least one of the one or more articulation pull wires coupled thereto, the pulley being rotatable about an axis by the robotic drive input, and a pulley brake configured to apply friction to the pulley to provide resistance to rotational movement of the pulley.

### BACKGROUND

The present disclosure relates to robotic instrument control, such as in robotic medical systems. Certain robotic medical procedures can involve the use of shaft-type instruments, such as endoscopes, which may be inserted into a patient and advanced to a target anatomical site. Such medical instruments can be articulatable, such that the tip and/or other portion(s) of the shaft can deflect in one or more planes using robotic controls. The ability to precisely control articulation of such instruments can affect procedural user experience and outcomes.

### SUMMARY

Described herein are systems, devices, and methods to facilitate instrument articulation control in connection with certain procedures, such as medical procedures. In particular, systems, devices, and methods in accordance with one or more aspects of the present disclosure can facilitate precise robotic control of articulation of an instrument shaft. For example, articulation of a shaft-type instrument can be performed using pull wires associated with the instrument shaft, wherein tensioning of the pull wire(s) causes deflection of an articulable tip of the shaft. The pull wires can be coupled to one or more respective pulleys actuatable by robotic drives. In some implementations, more predictable articulation response can be achieved by dampening shaft articulation action through the direct application of friction/resistance to one or more components of the articulation system. For example, a braking mechanism can be implemented that is configured to apply friction to one or more pull wire pulleys, which may be disposed within a base (e.g., handle) of the instrument from which the instrument shaft projects.

In some implementations, articulation dampening can be achieved by disposing a brake, such as an O-ring, between a pull wire pulley surface and a surface of an instrument base structure/housing. Such a brake mechanism can provide articulation dampening that improves articulation control. Static friction between the dampening brake (e.g., O-ring), the pulley, and/or the instrument base can be reduced through the application of grease or other lubricant between the brake and the contacting surface(s).

In some examples, friction can be adjustably and/or selectively applied to an articulation system component, such as a pull wire pulley, which can be implemented to compensate for component degradation or creep over time. Adjustable/selective friction application can further be implemented to provide active braking by varying articulation system (e.g., pulley) friction in real time for different articulation states or processes. For example, friction can be decreased during articulation to provide increased instrument sensitivity. In some implementations, friction can be increased in connection with a direction reversal to reduce the effects of articulation dead zones. For example, when changing pulley direction, the brake can be used to hold the articulation mechanisms in place while the motor traverses the articulation dead zone with increased motor speed, such that the articulation dead zone is less perceptible to the user. Articulation dampening/braking can be increased and decreased as needed to maintain a uniform level of force on the drive motor(s) throughout the change in pulley direction. In some implementations, increased articulation is implemented in relatively high articulation states and/or articulation is decreased in states of relatively low articulation.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an embodiment of a robotic medical system including a shaft-type instrument coupled to a robotic manipulator in accordance with one or more examples.

FIG. 2 illustrates medical system components in accordance with one or more examples.

FIG. 3 shows an exploded view of an instrument manipulator assembly including a shaft-type instrument having a tensioning pull wire pulley system and a robotic end effector in accordance with one or more examples.

FIG. 4 illustrates an articulatable shaft-type instrument and associated robotic manipulators in accordance with one or more examples.

FIG. 5 is a graph showing a relationship between pulley rotation and instrument deflection for an elastic instrument shaft in accordance with one or more examples.

FIG. 6 is a graph showing a relationship between pulley rotation and instrument deflection for a plastic instrument shaft in accordance with one or more examples.

FIG. 7A is a graph showing a relationship between pulley rotation and instrument deflection for a partially plastic and elastic instrument shaft in accordance with one or more examples.

FIG. 7B is a graph showing a relationship between pulley rotation and pull wire tension for a partially plastic and elastic instrument shaft in accordance with one or more examples.

FIG. 8 is a graph showing a relationship between pulley rotation and instrument deflection for a partially plastic and elastic instrument shaft implemented with articulation dampening in accordance with one or more examples.

FIG. 9 is a flow diagram for a process of controlling active articulation dampening for an instrument in accordance with one or more examples.

FIGS. 10A and 10B show perspective and side cross-sectional views, respectively, of an instrument base including a pulley brake in accordance with one or more examples.

FIG. 11 shows an instrument base with a rotation-actuated brake in accordance with one or more examples.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” “lateral,” and similar terms, are used herein to describe a spatial relationship of one device/element or anatomical structure to another device/element or anatomical structure, it is understood that these terms are used herein for ease of description to describe the positional relationship between element(s)/structures(s), such as with respect to the illustrated orientations of the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another. In some contexts features associated with separate figures that are identified by common reference numbers are not related and/or similar with respect to at least certain aspects.

The present disclosure provide systems, devices, and methods for dampening articulation of an instrument shaft, such as a medical endoscope, in a manner as to increase predictability and intuitiveness of robotic control of articulation mechanism(s). Articulation of instruments in accordance with the present disclosure can be implemented by tensioning one or more tendons, referred to herein as “pull wires,” that traverse a shaft of the instrument. The term “pull wire,” as used herein, can refer to any type of cable, cord, strand, tendon, filament, rod, band, tether, wire, string, fiber, chain, line, strap, tape, tube, lead, ribbon, or the like, configured to transmit force from an articulation control driver/actuator to cause articulation of a shaft. With respect to medical instruments described in the present disclosure, the term “instrument” is used according to its broad and ordinary meaning and may refer to any type of tool, device, assembly, system, subsystem, apparatus, component, or the like. In some contexts herein, the term “device” may be used substantially interchangeably with the term “instrument.” Furthermore, the term “shaft” is used herein according to its broad and ordinary meaning and may refer to any type of elongate cylinder, tube, scope (e.g., endoscope), prism (e.g., rectangular, oval, elliptical, or oblong prism), wire, or similar, regardless of cross-sectional shape. It should be understood that any reference herein to a “shaft” or “instrument shaft” can be understood to possibly refer to an endoscope.

### Robotic Medical Systems and Procedures

Robotically articulable instruments in accordance with the present disclosure can be utilized in connection with various medical procedures, such as kidney stone removal procedures. Advantageously, aspects of the present disclosure relate to systems, devices, and methods for dampening robotically controlled articulation of instrument shafts (e.g., endoscope shafts) in a manner as to temper the effects of jerking/jumping due to the breaking of static friction of a tensioning pulley mechanism. Furthermore, occurrences of elastic springing through backlash in articulation ‘dead zones’ can be reduced or eliminated by implementing articulation dampening as described herein.

During certain procedures, medical instrument(s), such as robotically controlled medical instrument(s) (e.g., endoscopes, access sheaths, working instruments), is/are inserted into a subject (e.g., a patient's body) and articulated or otherwise controlled. Within the subject, the instrument(s) may be positioned within a luminal network or other anatomy of the patient. As used herein, the term “luminal network” refers to any cavity structure within the body, whether comprising lumens or branches (e.g., a plurality of branched lumens, as in the lungs or blood vessels) or a single lumen or branch (e.g., within the urinary or gastrointestinal tracts). Instruments associated with aspects of the present disclosure can include, for example, any type of endoscope (i.e., “scope”), such as a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on.

Although certain aspects of the present disclosure are described in detail herein in the context of endoscopy procedures, such as ureteroscopy procedures, it should be understood that such context is provided for convenience and clarity, and instrument navigation concepts disclosed herein are applicable to any suitable medical procedures, such as various types of robotic medical procedures. For example, articulation dampening as disclosed herein may be implemented in connection with robotic ureteroscopy, bronchoscopy, laproscopy, arthroscopy, colonoscopy, laryngoscopy, neuroendoscopy, proctoscopy, anoscopy, gastroscopy, sigmoidoscopy, thoracoscopy, colposcopy, esophagoscopy, or other endoscopic or elongate-shaft-based procedure. It should be understood that any methodology described can be used in other contexts, such as animals, simulators, models of anatomy, cadavers, etc.

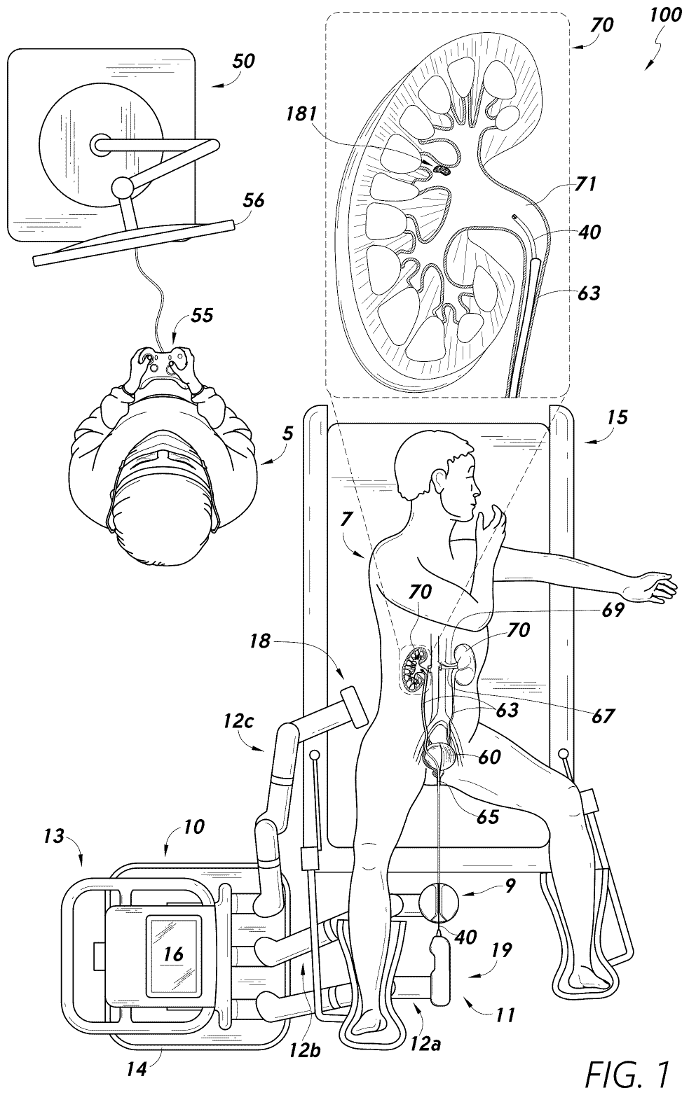

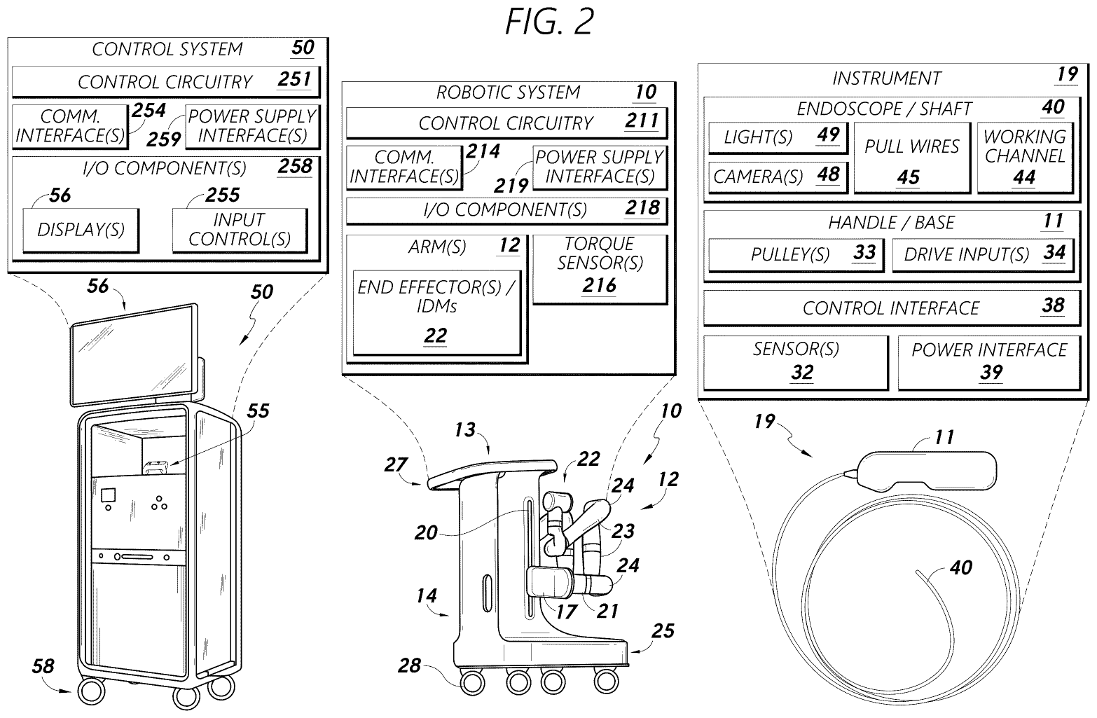

FIG. 1 illustrates an example medical system 100 for performing various procedures in accordance with aspects of the present disclosure. FIG. 2 shows detailed example implementations of certain components of the medical system 100 shown in FIG. 1. The description below of the medical system 100 and various components (e.g., robotic components) thereof may be understood with reference to either or both of FIGS. 1 and 2. Generally, robotic solutions can provide relatively higher precision, superior control, and/or superior hand-eye coordination with respect to control of certain instruments compared to strictly-manual solutions.

The medical system 100 includes a robotic system 10 configured to engage with and/or control an instrument 19 (e.g., endoscope/ureteroscope) including a proximal base 11 (e.g., handle) and a shaft 40 coupled to the base 11 at a proximal portion thereof. The robotic system 10 can be configured to facilitate execution of a medical procedure and can be arranged in a variety of positions and configurations, depending on the procedure. The medical system 100 can include a table 15 configured to hold the subject patient 7.

The robotic system 10 can include one or more robotic arms 12 configured to engage with and/or control the instrument 19 to perform one or more aspects of a procedure. As shown, each robotic arm 12 can include multiple arm segments 23 coupled to joints 24, which can provide multiple degrees of movement/freedom. The robotic system 10 can have an instrument feeder instrument 9 coupled to an arm 12b thereof to facilitate robotic advancement of the instrument 19. Another arm 12a may have the instrument base 11 coupled thereto.

The robotic system 10 can be electrically and/or communicatively coupled to any component of the medical system 100, such as to a control system 50, the table 15, an electromagnetic (EM) field generator 18, and/or the instrument 19. Robotic system 10 also includes a power supply interface 219, which may receive power to drive robotic system 10 via wire, battery, and/or any other suitable kind of power source. In addition, robotic system 10 one example includes various input/output (I/O) components 218 configured to assist the operator 5 or others in performing a medical procedure.

In some examples, robotic system 10 may be communicatively coupled with control system 50 via communication interfaces 214, 254. For example, the robotic system 10 may be configured to receive control signals from the control system 50 to perform certain operations, such as to position one or more of the robotic arms 12, manipulate (e.g., advance, articulate) the instrument 19, and so on. In response, the robotic system 10 can control, using certain control circuitry 211, actuators 217, and/or other components of the robotic system 10, to perform the operations. For example, the control circuitry 211 may control articulation of the shaft of the instrument 19 by actuating drive output(s) of the end effector 22 coupled to the instrument base 11. In some embodiments, the robotic system 10 and/or control system 50 is/are configured to receive images and/or image data from the instrument 19 representing internal anatomy of the patient 7.

The robotic system 10 can include a support structure 14 (also referred to as a “column”), a robotic system base 25, and a console 13 at the top of the column 14. The console 13 also includes a handle 27 to assist with maneuvering and stabilizing the robotic system 10. The column 14 may include one or more arm supports 17 (also referred to as a “carriage”) for supporting the deployment of the one or more robotic arms 12 (three shown in FIG. 1). The robotic arms 12 of the present example generally comprise robotic arm bases 21 and end effectors 22, separated by a series of linking arm segments 23 that are connected by a series of joints 24, each joint comprising one or more independent actuators. Each actuator 217 may comprise an independently controllable motor. The arm support 17 may be configured to vertically translate along the column 14. In some embodiments, the arm support 17 can be connected to the column 14 through slots 20 that guide the vertical translation of the arm support 17. The robotic system base 25 can include wheel-shaped casters 28.

The console 13 can provide both a user interface for receiving user input and a display 16 (e.g., screen or a dual-purpose device such as, for example, a touchscreen) to provide the physician/user 5 with both pre-operative and intra-operative data. Potential pre-operative data on the console/display 16 or display 56 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse.

Articulation of the shaft 40 of the instrument 19 may be controlled robotically, such as through operation of a robotic manipulator of the robotic system 10. The combination of the end effector 22 and the instrument base 11, as well as any intervening mechanics or couplings (e.g., sterile adapter), can be referred to as a manipulator assembly. The term “end effector” is used herein according to its broad and ordinary meaning and may refer to any type of robotic manipulator device, component, and/or assembly. In implementations in which an adapter, such as a sterile adapter, is coupled to a robotic end effector or other robotic manipulator, the term “end effector” may refer to the adapter (e.g., sterile adapter), or any other robotic manipulator device, component, or assembly associated with and/or coupled to the end effector. Furthermore, the terms “manipulator,” “robotic manipulator,” and “robotic manipulator assembly” are used according to their broad and ordinary meanings, and may refer to a robotic end effector and/or sterile adapter or other adapter component coupled to the end effector, either collectively or individually. For example, the terms “robotic manipulator” and “robotic manipulator assembly” may refer to one or more drive outputs, rails, arms, pulleys, gears, couplings, belts, guides, or the like, whether embodied in a robotic end effector, sterile adapter, and/or other component(s). Robotic manipulators of the present disclosure can include connectors to transfer pneumatic pressure, electrical power, electrical signals, and/or optical signals between the robotic system 10 and a coupled instrument, such as the instrument 19.

The control system 50 can be configured to interface with the robotic system 10, provide information regarding the procedure, and/or perform a variety of other operations. In some examples, the control system 50 can include wheel-shaped casters 58. The control system 50 includes a power supply interface 259, which may receive power to drive control system 50 via wire, battery, and/or any other suitable kind of power source. A control circuitry 251 of the control system 50 may provide signal processing and execute control algorithms to achieve the functionality of the medical system 100 as described herein.

The control system 50 can include various input/output (I/O) components 258 configured to assist the physician 5 or others in performing a medical procedure. For example, the I/O components 258 can be configured to allow for user input to control/navigate the instrument 19 and/or other robotically controlled instrument within the patient 7. In some examples, the physician 5 can provide input to the control system 50 and/or robotic system 10 via one or more input controls 255, wherein in response to such input, control signals can be sent to the robotic system 10 to manipulate the instrument 19. The control system 50 can receive real-time images that are captured by the instrument 19 and display the real-time images via the display(s) 56. Additionally, or alternatively, the control system 50 can receive signals (e.g., analog, digital, electrical, acoustic/sonic, pneumatic, tactile, hydraulic, etc.) from a medical monitor and/or a sensor associated with the patient 7, and the display(s) 56 can present information regarding the health or environment of the patient 7.

The system 100 can include certain control circuitry configured to perform certain of the functionality described herein, including the control circuitry 211 of the robotic system 10 and the control circuitry 251 of the control system 50. That is, the control circuitry of the system 100 may be part of the robotic system 10, the control system 50, or some combination thereof. Therefore, any reference herein to control circuitry may refer to circuitry embodied in a robotic system, a control system, or any other component of a medical system. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

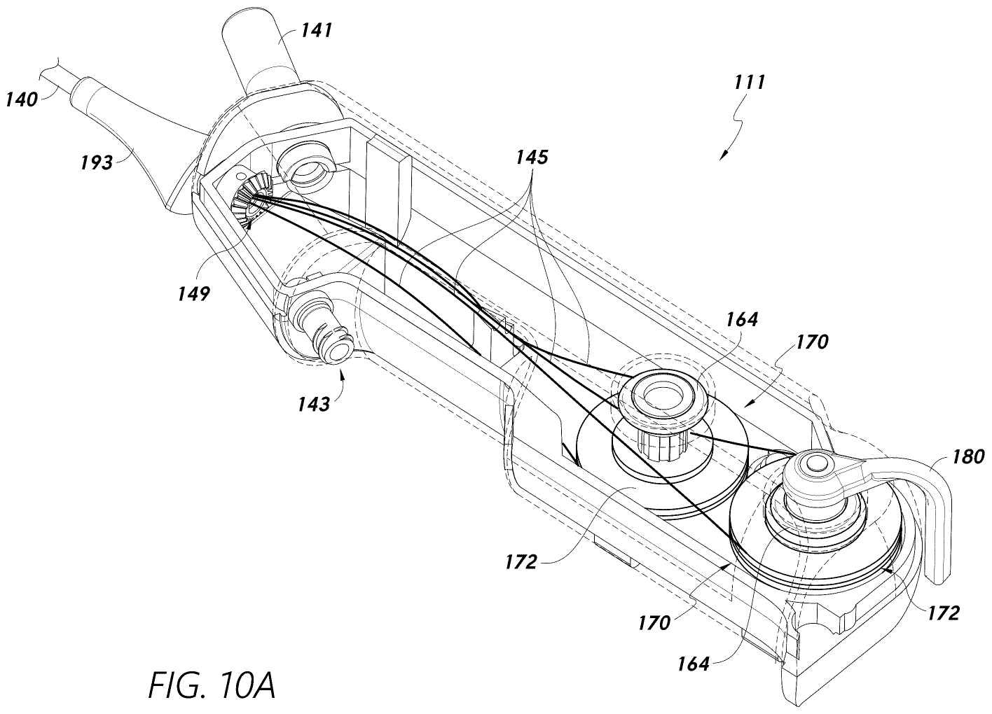

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211,251 may be entirely locally maintained/disposed or may be remotely located at least in part (e.g., communicatively coupled indirectly via a local area network and/or a wide area network). Any of the control circuitry 211, 251 may be configured to perform any aspect(s) of the various processes disclosed herein, including the processes shown in FIG. 9, as described below.

The control circuitry 211 and/or control circuitry 251 may be communicatively coupled to one or more torque sensors 216 configured to generate signals indicative of torque on one or more actuators of the robotic system 10, such as articulation pulley drive gears/outputs. The torque sensor(s) 216 may have any suitable or desirable configuration. For example, the torque sensor(s) 216 can act as a sensed mounting structure or load cell. In some embodiments, the torque sensor(s) 216 is/are configured as a reactive torque sensor that measures torque induced strain using one or more self-contained strain gauges to create a load cell. Although torque sensors 216 of a robotic system are described herein in the context of determining force on pull wire pulleys of a steerable instrument, such references may be understood to represent any type of sensor(s) or sensing mechanism configured to generate signals indicative of forces on pull wires and/or associated pulleys/actuators, such as strain gauges or the like. References herein to torque sensors or other types of force sensors (e.g., strain gauges) can be any type of sensor configured to measure force/load on a robotic actuator, whether such force is rotational or linear in nature. That is, although rotational robotic output drives are disclosed in some contexts herein, it should be understood that inventive concepts disclosed herein apply to other types of actuators, such as linear drives.

As noted above, robotic system 10 may be communicatively coupled with control system 50 via communication interfaces 214, 254. The various components of the system 100 can be communicatively coupled to each other over a network, which can include a wireless network and/or a wired network. Example networks include one or more personal area networks (PANs), local area networks (LANs), wide area networks (WANs), Internet area networks (IANs), cellular networks, the Internet, personal area networks (PANs), body area network (BANs), etc. Furthermore, in some embodiments, the various components of the systems can be connected for data communication, fluid exchange, power exchange, and so on via one or more support cables, tubes, or the like.

The control system 50 and/or the robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to the respective control circuitry. In some embodiments, the user may engage the user controls 55 to command robotic shaft articulation, as described herein.

The instrument 19 (e.g., endoscope) includes a base 11 (e.g., handle) coupled to an elongate shaft 40. The shaft 40 of the instrument 19 can include one or more lights 49 and one or more cameras or other imaging devices 48. The shaft 40 can further include one or more working channels 44, which may run a length of the shaft 40. The instrument 19 can be powered through a power interface 39 and/or controlled through a control interface 38, each or both of which may interface with a robotic end effector of the robotic system 10. The instrument 19 may further comprise one or more sensors 32, such as pressure sensors and/or other force-reading sensors, which may be configured to generate signals indicating position and/or forces experienced at/by one or more components of the instrument 19.

The instrument 19 includes certain mechanisms for causing the shaft 40 to articulate/deflect with respect to an axis thereof. For example, the instrument 19 may include one or more drive inputs 34 associated with one or more pulleys 33 that are configured to tension/untension pull wires 45 to cause articulation of the shaft 40.

In an example use case, if the patient 7 has a kidney stone (or stone fragment) 181 located in a kidney 70, the physician 5 may perform a procedure to remove the stone 181 through the urinary tract (63, 60, 65). In some embodiments, the physician 5 can interact with the control system 50 and/or the robotic system 10 to cause/control the robotic system 10 to advance and navigate the instrument shaft 40 (e.g., a ureteroscope) from the urethra 65, through the bladder 60, up the ureter 63, and into the renal pelvis 71 and/or calyx network of the kidney 70 where the stone 181 is located. The physician 5 can further interact with the control system 50 and/or the robotic system 10 to cause/control the advancement of a basketing device or other instrument through a working channel of the instrument shaft 40 to facilitate capture and removal of a kidney stone or stone fragment.

### Robotic Instrument Articulation

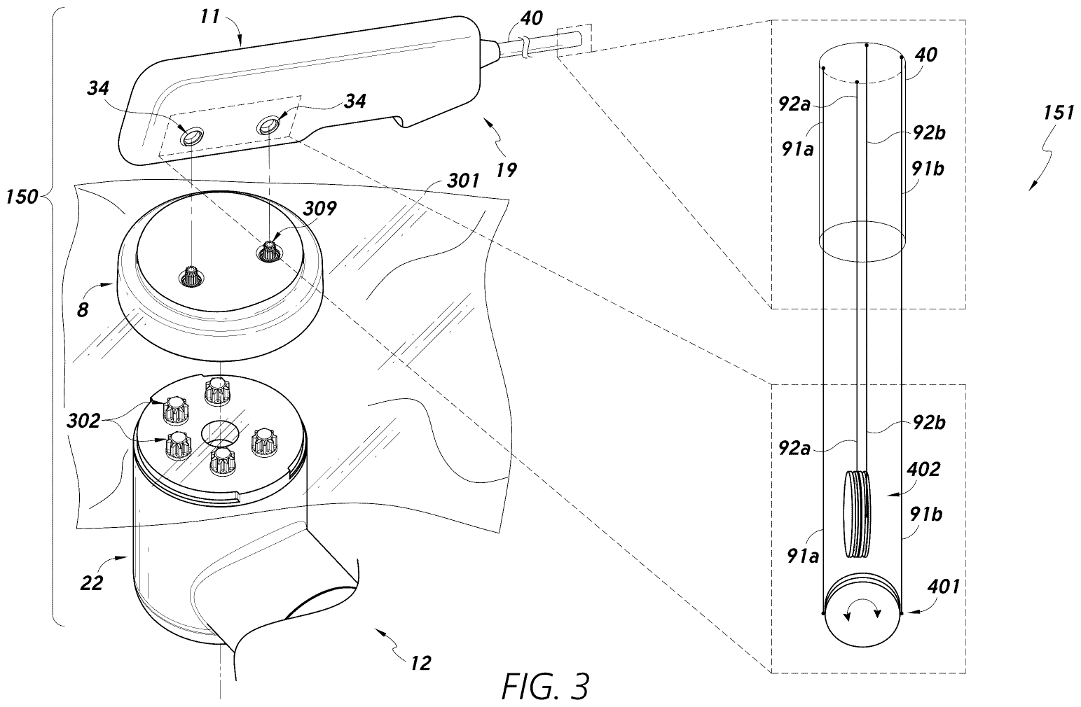

FIG. 3 shows an exploded view of an instrument manipulator assembly 150 including an instrument 19 having a pull wire tensioning pulley system 151 for articulating a shaft 40 of the instrument 19, as well as a robotic end effector 22. The instrument manipulator assembly 150 can further include an adapter component 8 mountable to the end effector 22 and configured to provide a driver interface between the end effector 22 and the instrument base 11. The adapter 8 and/or the instrument base 11 may be removable or detachable from the robotic arm 12 and may be devoid of any electro-mechanical components, such as motors, in some embodiments. A drape 301 may be coupled to the adapter 8 in such a way as to allow for translation of mechanical torque from the end effector 22 to the adapter 8. The adapter 8 may generally be configured to maintain a seal around the actuating components thereof, such that the adapter 8 provides a sterile barrier itself. With the arm 12 draped in plastic, the physician and/or other technician(s) may interact with the arm 12 and/or other components of the robotic cart (e.g., screen) during a procedure, while the surgical area remains protected from contamination. In some embodiments, the adapter 8 can include connectors to transfer pneumatic pressure, electrical power, electrical signals, and/or optical signals from the robotic arm 12 and/or end effector 22 to the instrument base 11. The end effector 22 can include drive outputs 302 (e.g., drive splines, gears, or rotatable disks with engagement features) to control/articulate a medical instrument.

The instrument base 11 can include a plurality of drive inputs 34 in fixed positions spaced apart along a mating surface of the instrument base 11 that couple/engage with corresponding ones of the drive outputs 302 of the end effector 22. A mechanical assembly within the instrument base 11 can allow the drive inputs 34 to be used to drive articulation of the shaft 40. Each drive input can comprise a receptacle configured to mate with a drive output that is configured as a spline. Thus, the drive outputs 302 can be rotated to cause corresponding rotation of the drive inputs 34 to control functionality of the instrument base 11.

References herein to an “instrument device manipulator assembly,” “instrument manipulator assembly,” “manipulator,” “manipulator assembly,” as well as other variations thereof, can refer to any subset of the components of the assembly 150 shown in FIG. 3. Furthermore, it should be understood that references herein to an “actuator” can refer to any component of the assembly 150 of FIG. 3 that affects or causes, either directly or indirectly, movement of an instrument/component engaged with, coupled to, or otherwise actuatable by, a component of the assembly 150. For example, an “actuator” may comprise any set or subset of the following devices or components: adapter drive output(s), adapter drive input(s), pulleys, belts, gears, pegs, pins, end effector drive output(s), and/or structures and/or control circuitry configured to cause actuation of the same. An actuator may be any component, device, or structure configured such that movement thereof causes corresponding movement in another component, device, or structure, whether integrated with or separate from the actuator.

The pulley system 151 can include one or more pulleys 401, 402, which may each include one or more pulley wheels (e.g., two pulleys as shown). Each pulley can be robotically controlled/rotated by a robotic drive output (e.g., the drive output 309). For example, the pulley 401 can be configured to rotate about in a primary articulation plane, while the pulley 402 can have a rotational axis that is perpendicular to the primary articulation plane The base 11 can employ any known mechanisms to convert a torque/force received from rotational axes of the drive outputs 302 to the rotational axes of the pulleys 401, 402.

A first set of pull wires (e.g., separate wires, or separate segments of a common wire) 91a, 91b can be attached to the first pulley assembly 401. In some embodiments, the first set of pull wires 91a, 91b can be coupled to and/or emanate from opposite sides of the pulley assembly 401, such that rotation of the pulley assembly 401 increases tension in one of the pull wires 91a, 91b and decreases tension in the other, depending on the direction or rotation. The pull wires 91a, 91b may be attached to (e.g., wound on) a single pulley wheel, or to separate pulley wheels rotationally fixed to one another, as shown. Similarly, a second set of pull wires 92a, 92b can be connected to the second pulley assembly 402.

A distal end/tip of the shaft 40 can be coupled to ends of the pull wires 91a, 91b, 92a, 92b. More specifically, the pull wires 91a and 91b can be coupled to opposing sides of the distal end of the shaft 40 in the primary articulation plane, whereas the pull wires 92a and 92b can be coupled to opposing sides of the distal end of the shaft 40 in the secondary articulation plane. At least a distal articulation portion of the shaft 40 can be articulated based on rotation of the pulley assemblies 401, 402.

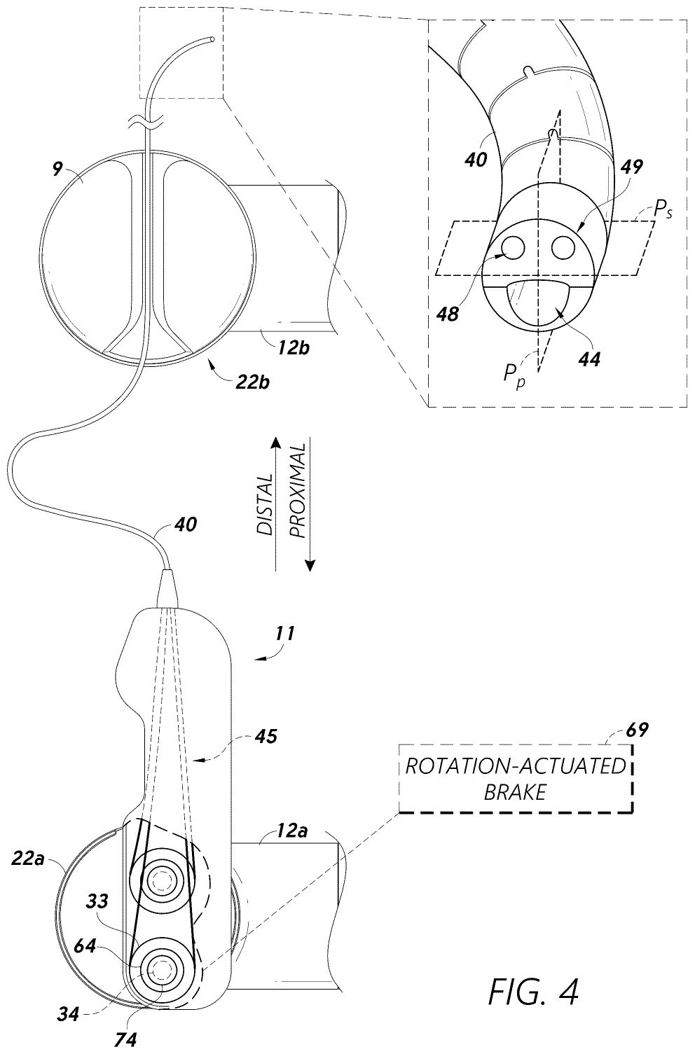

FIG. 4 provides additional views and instrumentation associated with an articulable instrument 19 to further demonstrate robotic shaft articulation control in accordance with aspects of the present disclosure. The shaft 40 of the instrument 19 can accommodate wires and/or optical fibers to transfer signals to/from an optical assembly at a distal end 42 of the instrument shaft 40, which can include one or more imaging devices 48, such as optical camera(s). The instrument shaft 40 can further include one or more light sources 49, such as LED or fiber-optic light source(s)/lens(es).

In some embodiments, the shaft 40 is configured to be articulated with, for example, six degrees of freedom, including XYZ coordinate movement, as well as pitch, yaw, and roll. The instrument base 11 can be coupled to an end effector 22a of a robot arm 12a (or rail, etc.) and can manipulate the instrument shaft 40 using one or more pull wires 45 coupled to the distal end 42 of the shaft 40. The shaft 40 may exhibit nonlinear behavior in response to forces applied by the pull wires 45. The nonlinear behavior may be based on stiffness and compressibility of the shaft 40, as well as variability in slack or stiffness between different elongate movement members.

The instrument shaft 40 can be deflectable in one or two directions within a first/primary plane Pₚ. The instrument shaft 40 can also be deflectable in one or two directions in a second/secondary plane Pₛ, which may be orthogonal to the primary plane Pₚ. Although the primary Pₚ and secondary Pₛ deflection planes are shown in a particular configuration, it should be understood that the illustrated secondary plane Ps may be the primary plane Pₚ and vice versa.

The instrument base 11 can include a plurality of drive inputs 34, each associated with a respective pull wire articulation pulley 33. The plurality of pull wires 45 can be coupled to the plurality of pulleys 33 and extend along the shaft 40. The plurality of drive inputs 34 can be configured to control or apply tension to the plurality of pull wires 45 in response to rotation of drive outputs of the coupled robotic system.

The instrument base 11 includes one or more pulley brakes 64 configured to dampen articulation of the shaft 40 as the pulley(s) 33 are rotated/driven. Such articulation dampening can produce more predictable intuitive control of the robotic articulation mechanism. For example, when initiating rotation of a pulley 33, or changing direction of pulley rotation, it may be necessary to first overcome static friction of the pulley 33, such as may be associated with frictional contact between the pulley and one or more surfaces of the instrument base 11. When static friction is overcome by the torque of the robotic drive output, jumps/jerks in pulley rotation may result as the rotational motion of the pulley 33 accelerates forward due to the application of force at a level necessary to overcome the static friction. The implementation of the pulley brake(s) 64 can introduce friction between the pulley 33 and the instrument base 11 that resists jumps/jerks in pulley rotation as static friction is overcome.

Implementation of the pulley brake(s) 64 can further reduce or eliminate the effects of elastic springing through backlash when articulating the instrument shaft 40. For example, as described in greater detail below, “backlash” can refer gaps/slack between the robotic drive output used to control articulation of the instrument 19 and the drive input and/or pulley mechanics of the instrument base 11. Such gaps/slack can cause a delay, or “dead zone,” in the articulation of the instrument shaft 40 when the direction of movement of the relevant pulley 33 is reversed. As an example, small spaces may be present between the teeth of the drive output gear, such that it may be necessary for the gear to rotate some small amount before the drive output fully engages and rotates the pulley 33, which may be coupled to or integrated with the drive input receptacle of the instrument base 11. The space between the gear teeth may be considered backlash. In the context of articulating the instrument shaft 40, backlash can cause a slight delay or slack before the instrument responds to control commands, particularly when changing direction. For example, when the shaft 40 is deflected one way and then switched to deflect in the opposite direction, the shaft 40 may not move immediately due to the gap between the moving parts, until the drive output has rotated through the backlash range. Delay from backlash can make the movement of the instrument less smooth and precise, making it harder for the operator to accurately control the instrument. The pulley brake(s) 64 can impede elastic spring of the pulley(s) 33, which may otherwise result due to the drive output moving relatively quickly and/or freely through the backlash range of motion and abruptly engaging the pulley when reaching the end of the range. That is, when the drive output reaches the end of the backlash range, the brake(s) 64 can advantageously impede quick jumps in the pulley rotation, providing smoother articulation and control coming out of dead zones.

Implementation of the pulley brake(s) 64 can further allow for simplified control algorithms for controlling articulation of the shaft 40. For example, the pulley brake(s) 64 can increase the plasticity of the shaft 40, providing bend-and-stay behavior, such that, when changing directions, the backlash/dead-zone range need only be crossed once. Therefore, where the amount of backlash for a given instrument manipulator assembly is known, implementations of the present disclosure can improve control and user experience when articulating an instrument by automatically causing the drive output to speed through the dead zone in a manner that is less perceivable by the user.

Pulley braking using any braking mechanism of the robotic system (e.g., brake(s) 64, 69) can be implemented to apply friction in a selective manner. For example, pulley braking can be implemented using a closed-loop control process, wherein control circuitry of the system may be configured to determine/detect the presence of articulation dead zones, wherein braking can be selectively increased in such dead zones. Dead zone determination/detection can be implemented using instrument position sensors, such as electromagnetic field generators and sensors, to track the real-time position of the tip of the instrument shaft. In some examples, shape sensing light fiber may be used to determine real-time position and/or shape of the instrument shaft.

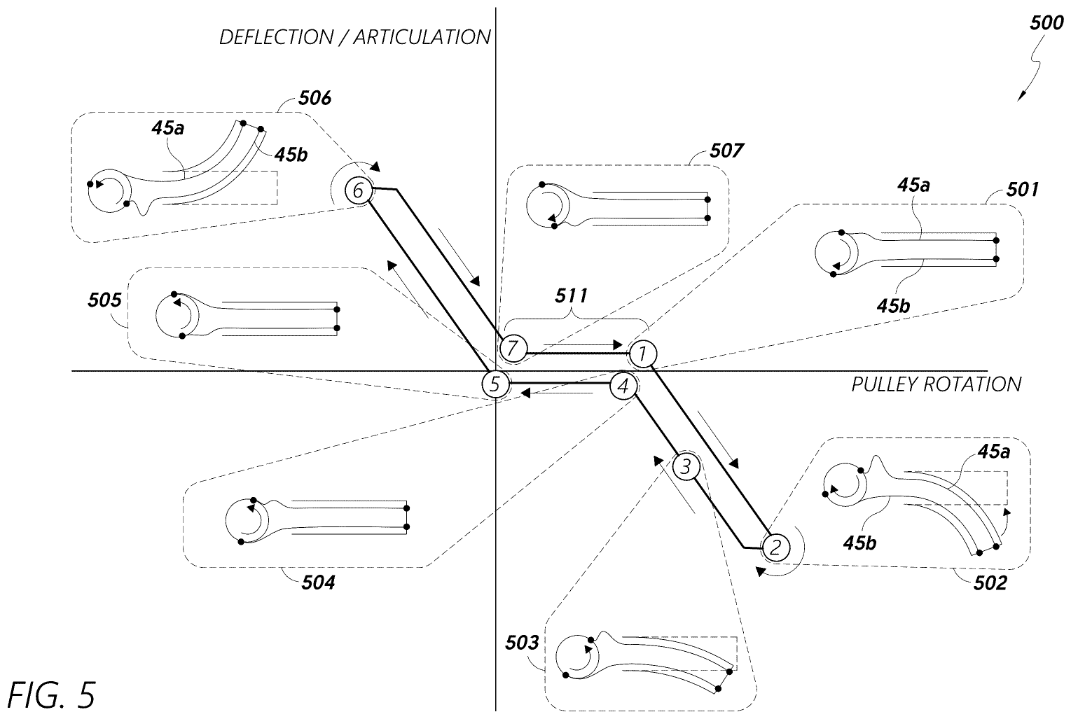

FIG. 5-8 include graphs showing relationships between pulley rotation and instrument deflection/articulation. Each of the graphs are plotted on a plane having an X-axis that represents pulley rotation (e.g., degree/amount of rotational relative to a reference frame) and a Y-axis that represents deflection/articulation (e.g., inclination) of an instrument. The center line on the X-axis can indicate neutral rotational position (i.e., zero rotation), or an arbitrary position, of a pulley, or any other rotational position. Moving toward the right of the center line along the X-axis can indicate increased clockwise rotation while moving toward the left can indicate increased clockwise rotation. The center line on the Y-axis can indicate zero inclination of the tip, such that the instrument shaft is generally straight. With this understanding, each of the relationships will be described in a clockwise traversal of the articulation profile. Each starting coordinate and the clockwise traversal are selected solely to facilitate the following descriptions and may be deemed arbitrary.

FIG. 5 is a graph 500 showing an example relationship between pulley rotation and instrument articulation for an elastic instrument shaft. Elastic instrument shafts can be considered shafts that exhibit a tendency to return to a pre-articulated position when articulated, such as when articulation tension is removed or reduced. An example of an elastic instrument shaft can be a shaft that is springy and returns to a neutral position (e.g., zero-degree articulation) on its own. The relationship between articulation and pulley rotation with respect to the graph 500 is described below moving along the graph in a clockwise manner, from a first state (corresponding to point (1) and image 501) of the pulley and instrument to a second state (corresponding to point (2) and image 502), a third state (corresponding to point (3) and image 503), a fourth state (corresponding to point (4) and image 504), a fifth state (corresponding to point (5) and image 505), a sixth state (corresponding to point (6) and image 506), a seventh state (corresponding to point (7) and image 507), and returning to the first state (1). The various states shown and described represent configurations and/or conditions of pulley, pull wire, and instrument shaft elements of a robotically articulable instrument.

The first state (1), shown in image 502, represents a clockwise (e.g., positive) pulley rotation, with zero instrument articulation. At the first state (1), a first pull wire 45a has zero, or an insubstantial amount of, tension while the opposite pull wire 45b is taut due to the clockwise pulley rotation up to the tension interface/threshold of the pull wire 45b. Therefore, further rotation beyond the position (1) will result in articulation in the direction of the side associated with the pull wire 45b. The range 511 of rotation leading to state (1) that does not result in instrument articulation can be considered a ‘dead zone.’

The second state (2), shown in image 502, represents an increased clockwise pulley rotation relative to the state (1), resulting in articulation in the downward direction with respect to the orientation of the graph 500 in FIG. 5. Between state (1) and state (2), an increase in tension on the pull wire 45b causes a linear increase in the downward articulation. While the pull wire 45b has increased tension compared to the state (1), the pull wire 45a remains with zero or an insubstantial amount of tension.

The third state (3), shown in image 503, represents an application of some amount of counterclockwise pulley rotation relative to articulated state (2). Here, as the shaft is elastic and exhibits a tendency to return to the neutral position, the pull wire 45b is taut and resists, to some degree, the tendency of the shaft to pull back towards the neutral/straight position. The downward articulation continues to be proportional to the degree of clockwise pulley rotation between the rotation positions (2) and (4) and the associated tension on the pull wire 45b. The pull wire 45a has zero or insubstantial tension in state (3), shown in image 503.

The fourth state (4), shown in image 504, and the fifth state (5), shown in image 505, represent configurations over the range 511 of pulley rotations corresponding to a neutral/straight position of the instrument shaft. Between states (4) and (5), there is generally insufficient tension on either of the pull wires 45a, 45b to overcome the elastic tendency of the instrument shaft, such that the shaft remains at the neutral position. That is, the pulley rotation within the range 511 may not provide a threshold tension level required to cause the shaft tip to articulate. For example, the pull wires 45a, 45b may provide zero or some insubstantial tension in the range 511, such that the elastic tendency of the instrument shaft fully controls the positioning of the tip of the shaft. In any event, the shaft tip will only articulate in the upward direction (with respect to the orientation of the graph 500 shown in FIG. 5) when counterclockwise pulley rotation to the left of state (5) is applied, and will only articulate in the downward direction when clockwise pulley rotation to the right of the state (1) is applied. Referring again to the clockwise traversal of the graph 500, at state (5), a counterclockwise pulley rotation has not yet caused upward articulation. As pulley rotation in the range 511 between state (4) and state (5) does not result in a change in articulation of the instrument shaft, the range 511 can be considered a ‘dead zone.’

The sixth state (6), shown in image 506, represents an increased counterclockwise pulley rotation relative to the position of state (5), causing an upward articulation that mirrors that of the state (2), but in the opposite direction. Between states (5) and (6), an increase in tension on the pull wire 45a causes a linear increase in the upward articulation. The opposite pull wire 45b remains with zero or an insubstantial amount of tension.

Between state (6) and state (7), which is shown in image 507, clockwise pulley rotation is applied. Here, as the shaft is elastic and exhibits a tendency to return to the neutral position, the pull wire 45a is taut and resists the tendency of the shaft to pull back towards the neutral position, thereby maintaining a state of articulation of the instrument shaft. The upward articulation continues to be proportional to the total counterclockwise pulley rotation (e.g., the pulley rotation is to the left of the X-axis center line) and the tension on the pull wire 45a. In such range, the pull wire 45b has zero or insubstantial tension.

At the seventh state (7), the instrument shaft tip reaches the neutral position again due to its elastic tendency. From such position, until reaching the position of state (1), any clockwise pulley rotation does not produce sufficient tension in the pull wire 45b to overcome the elastic bias of the instrument shaft, and so the shaft remains at the neutral position in the dead zone 511. Between states (7) and (1), the elastic instrument shaft behaves in a similar manner as between states (4) and (5).

As demonstrated in the graph 500 and suggested in the description above, the elastic instrument shaft represented in FIG. 5 can exhibit at least two reversal/traversal zones/regions, namely between states (4) and (5) and between states (7) and (1), as identified by the range identifier 511, that exhibit unaltered articulations even when the pulley is rotated. The zones/ranges 511 are flat (or near flat) in the graph 500 due to their lack of changes in Y-axis articulation in response to changes in X-axis pulley rotation. These flat zones can be considered as ‘dead zones’ in which the elastic instrument shaft may remain unresponsive to some amount of pulley rotation.

In view of the graph 500 described above, perfectly elastic instruments can present various undesirable issues with respect to instrument articulation control. For example, as referenced above, the elastic instrument shaft may generally be unresponsive to pulley rotation changes while in reversal dead zones. The unresponsiveness of the instrument articulation in the dead zones may be based and/or caused at least in part by shaft stiffness and/or looseness of pull wires in terms of how the system components are manufactured, assembled, engaged, and/or used. Such mechanical conditions can further result in springy articulation that is challenging to control. Springy articulation can possibly require undesirably elevated level of tension to articulate the tip. Furthermore, articulation system components, such as pulley(s) or pull wires, may be more likely to break under elevated levels of tension resulting from compensation for springy articulation. Further, the stubbornness to articulate of such instruments can cause user frustration.

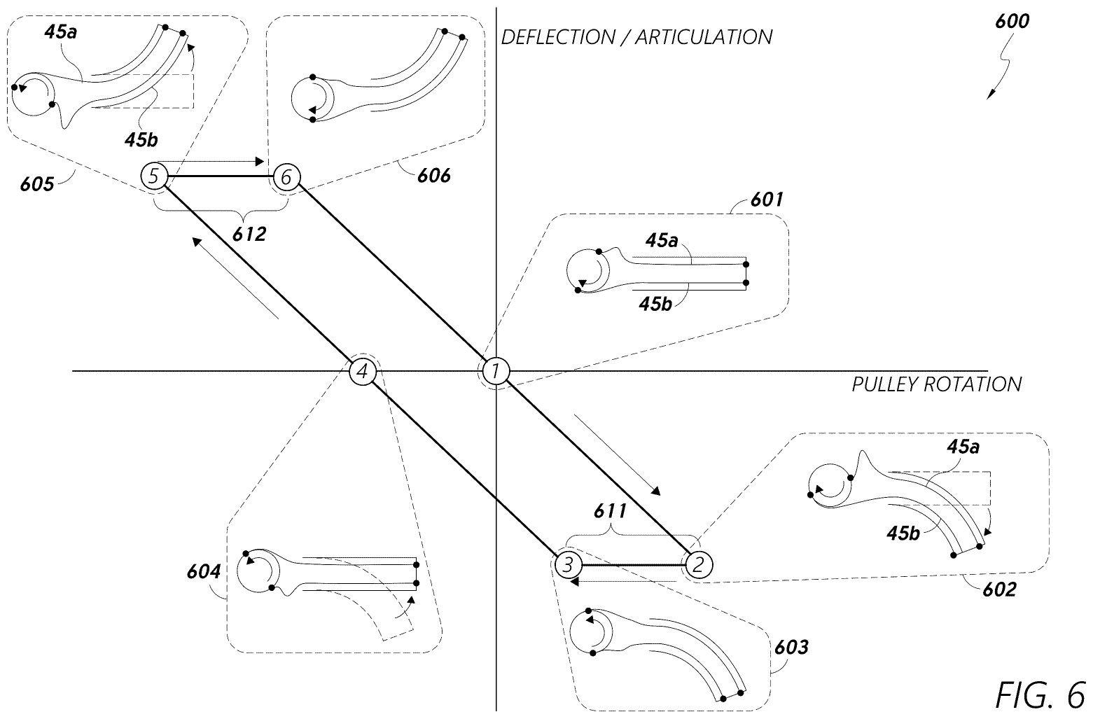

FIG. 6 is a graph 600 showing an example relationship between pulley rotation and instrument articulation for a plastic instrument shaft. The plastic instrument shaft can be a shaft that exhibits a tendency to remain articulated once manipulated/forced to an articulated position. An example of a plastic instrument shaft can be a shaft that is rigid or flaccid/limp and maintains its shape or position once adjusted rather than returning to a neutral position (e.g., zero degree articulation) on its own. The relationship between articulation and pulley rotation with respect to the graph 600 is described below moving along the graph in a clockwise manner, from a first state (corresponding to point (1) and image 601) of the pulley and instrument to a second state (corresponding to point (2) and image 602), a third state (corresponding to point (3) and image 603), a fourth state (corresponding to point (4) and image 604), a fifth state (corresponding to point (5) and image 605), a sixth state (corresponding to point (6) and image 506), and returning to the first state (1). The various states shown and described represent configurations and/or conditions of pulley, pull wire, and instrument shaft elements of a robotically articulable instrument.

The first state (1), shown in image 601, represents a clockwise (e.g., positive) pulley rotation and zero articulation. At the first state (1), a first pull wire 45a has zero or an insubstantial amount of tension, while the opposite pull wire 45b is taut based on the clockwise pulley rotation corresponding to the position of the first state (1). The second state (2), shown in image 602, represents an increased clockwise pulley rotation relative to state (1), resulting in downward articulation with respect to the illustrated orientation of the graph 600 of FIG. 6. Between state (1) and state (2), clockwise pulley articulation causes an increase in tension on the pull wire 45b, which in turn causes a linear increase in the downward articulation of the instrument shaft. While the pull wire 45b has increased tension in state (2) compared to state (1), the opposite pull wire 45a remains with zero or an insubstantial amount of tension.

The third state (3), shown in image 603, represents the configuration of the shaft at a pulley rotation position closer to neutral after counterclockwise rotation of the pulley from the position of state (2). For example, between state (2) and state (3), counterclockwise pulley rotation in connection with a change in articulation direction decreases tension on the pull wire 45b. However, the counterclockwise pulley rotation does not alter the downward articulation previously articulated at state (2) due to the plastic nature of the instrument shaft. As illustrated, the flat (e.g., parallel to the X-axis) response zone 611 between states (2) and (3) indicates a constant articulation configuration of the instrument shaft. The lack of articulation response during the traversal between states (2) and (3) may be observed when both of the pull wires 45a, 45b are loose (e.g., without meaningful tension) and, therefore, unable to adjust the articulation of the shaft. As the instrument shaft is plastic and does not return to its neutral position on its own, no change in articulation is observed during the traversal of the range/zone 611, which may be considered a dead zone.

When the traversal reaches state (3), shown in image 603, the pull wire 45a becomes taut based on the counterclockwise pulley rotation, such that the shaft is ready for a change of direction. The fourth state (4), shown in image 604, represents a counterclockwise (e.g., negative) pulley rotation to a point of reaching a zero articulation, or ‘neutral,’ shaft shape/configuration. At state (4), the pull wire 45a is taut, while the opposite pull wire 45b has zero or an insubstantial amount of tension based on the counterclockwise pulley rotation.

The fifth state (5), shown in image 605, is plotted at an increased counter-clockwise pulley rotation relative to state (4), which results in upward articulation with respect to the illustrated orientation of the graph 600 of FIG. 6. Between state (3) and state (5), an increase in tension on the pull wire 45a causes a linear increase in the upward articulation. The opposite pull wire 45b remains with zero or insubstantial amount of tension in such range/zone.

The sixth state (6), shown in image 606, represents the configuration of the instrument shaft after pulley rotation closer to the neutral, zero rotation pulley position. Between state (5) and state (6), clockwise pulley rotation brings the pulley rotation position back towards neutral, thereby decreasing tension on the pull wire 45a. However, the clockwise pulley rotation does not alter the upward articulation previously articulated in connection with state (5). As illustrated, the flat (e.g., parallel to the X-axis) articulation response between states (5) and (6) reflects the consistent articulation configuration of the shaft over such range/zone. The lack of articulation response during the traversal between state (5) and state (6) may be observed when both of the pull wires 45a, 45b loose (e.g., without meaningful tensions) and, therefore, unable to adjust articulation. As the plastic instrument shaft is rigid and/or limp in a manner as to not be biased towards the neutral straight shape of the shaft, no change in articulation is observed in the absence of pull wire tension. When the traversal reaches state (6), the pull wire 45b becomes taut based on the clockwise pulley rotation. Here, the shaft is primed to begin articulating back to the straight shape of the shaft, and further in the downward direction as the pulley is further rotated.

The plastic instrument shaft exhibits at least two traversal zones/ranges 611, 612, between states (2) and (3) and between states (5) and (6), that exhibit unaltered articulations even when supplied changes in pulley rotation. Such zones 611, 612 are flat (or near flat) in the graph 600 due to their lack of changes in the articulation Y-axis in response to changes in the pulley rotation X-axis. These flat zones can be considered as ‘dead zones’ in which the plastic instrument shaft may remain unresponsive to some amount of pulley rotation.

In view of the graph 600 described above, perfectly plastic instruments can present various undesirable issues with respect to instrument articulation control. For example, as referenced above, the plastic instrument shaft may generally be unresponsive to pulley rotation changes while in the dead zones. More specifically, the unresponsiveness may be caused by a combination of a shaft stiffness, or lack thereof, and/or by loose pull wires. Such mechanical conditions can result in a wobbly articulation behavior that arbitrarily articulates in some instances, which can cause user frustration. Further, it is also possible that pull wires degrade over time and eventually prematurely render the plastic instrument uncontrollable.

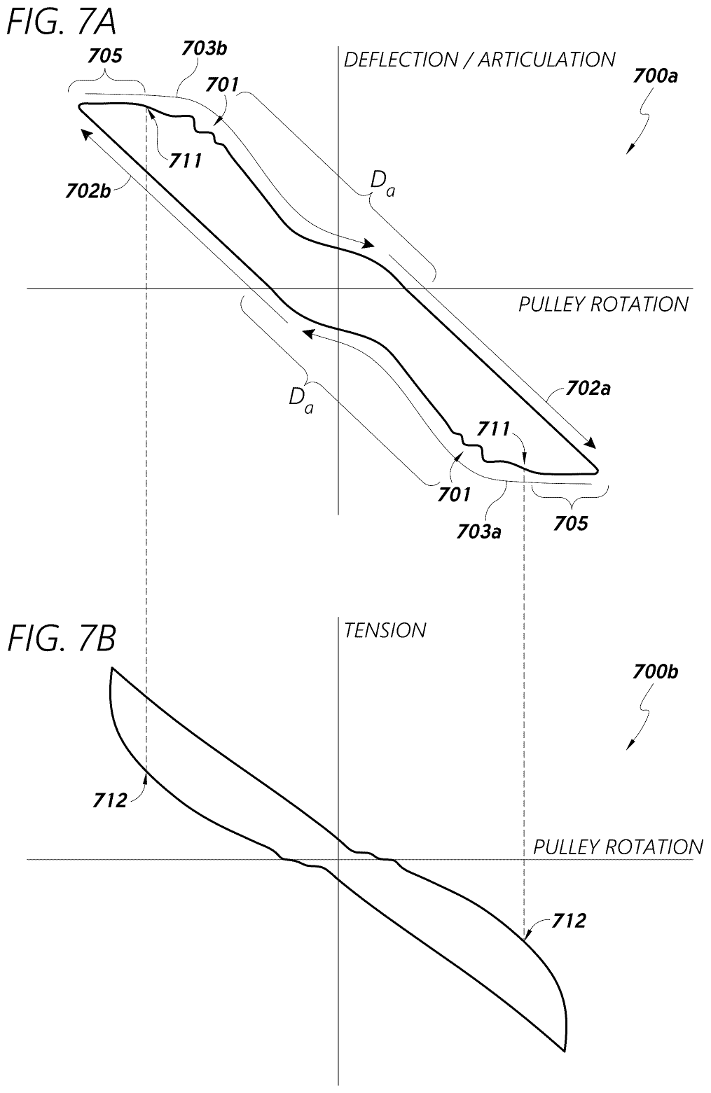

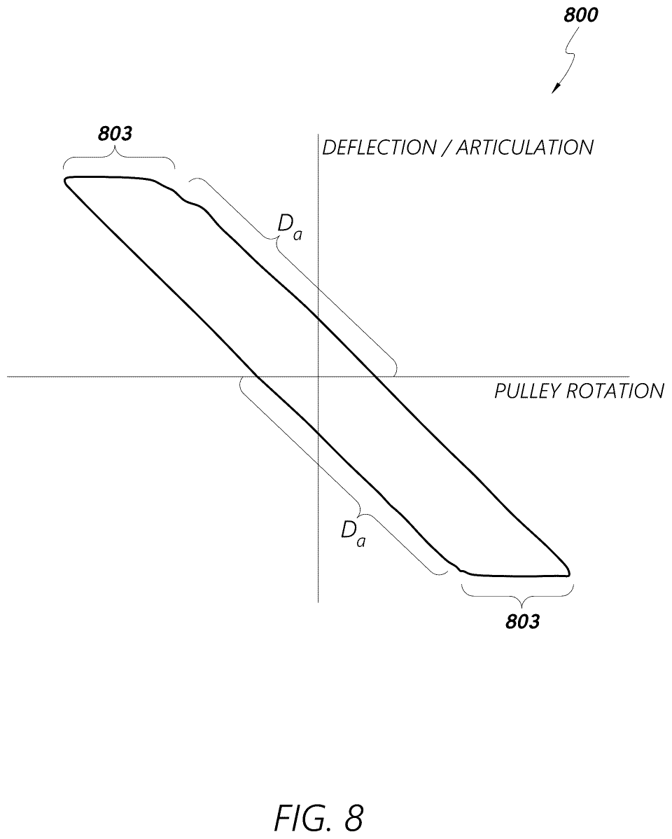

FIG. 7A is a graph 700a showing an example relationship between pulley rotation and instrument deflection for an instrument shaft that is partially plastic and partially elastic in nature in accordance with one or more embodiments. The instrument shaft represented by the graph 700a can correspond to a real-world implementation of an articulable instrument shaft. That is, while implementation/manufacturing of perfectly plastic or elastic instruments in a real-world setting may be unachievable, a hybrid plastic/elastic instrument shaft may be achievable. The hybrid instrument shaft can have a tendency, unlike a perfectly plastic instrument shaft, to return to its neutral position on its own, but to a lesser degree than for a perfectly elastic instrument shaft. In some cases, a hybrid instrument provides a middle ground between plasticity and elasticity more desirable than fully plastic or elastic shafts.

Unlike the rotation/articulation responses of fully elastic and plastic instruments as described in detail above, the rotation/articulation response of a hybrid instrument can generally include both linear, or mostly linear, zones 702a, 702b and nonlinear, or mostly nonlinear, zones 703a, 703b. The linear zones can include a clockwise linear zone 702a and a counterclockwise linear zone 702b. Within the linear zones 702a, 702b, pulley rotation causes a proportional shaft articulation represented by slopes of their respective lines.

The nonlinear zones can include a first nonlinear zone 703a and a second nonlinear zone 703b. As illustrated, the nonlinear zones 703a, 703b connect the clockwise linear zone 702a and the counterclockwise linear zone 702b. In other words, the instrument shaft can traverse the first nonlinear zone 703a when reversing its pulley rotational direction from clockwise to counter-clockwise (or vice versa). Similarly, the instrument shaft can traverse the second nonlinear zone 703b when reversing its pulley rotational direction from counter-clockwise to clockwise.

Due to the curve of the non-linear zones 703a, 703b, compared to a fully plastic instrument shaft, the hybrid instrument shaft can have less, or no, dead zone when reversing pulley rotation direction. Accordingly, the shaft of the hybrid instrument remains at least somewhat responsive to change in pulley rotation even when changing direction. Furthermore, as the hybrid instrument shaft is neither wobbly nor too stiff, it can reduce operator frustration and improve instrument durability.

The instrument shaft of the graph 700a may be representative of a possible real-world instrument shaft, and so may not have perfectly linear articulation response, particularly during de-articulation. For example, unpredictable stepping 701 may occur during de-articulation of the instrument shaft, as represented by the marked de-articulation zones Da. Such stepping 701 may occur as a result of backlash due to the slack or play between mechanical components of the articulation system, such as gears and pulleys. When the direction of pulley rotation changes, this slack must be taken up before effective (e.g., linear) motion occurs. Therefore, during de-articulation, backlash can cause a delay or non-linear response as the system transitions from one direction to another, resulting in jerky or unpredictable movements.

Static friction, or ‘stiction,’ represents frictional force that must be overcome to start moving the pull wire pulley(s) from rest, and can further produce nonlinear stepping 701. For example, when the instrument shaft starts de-articulating, the initial force needed to overcome static friction can cause a sudden jump or step, leading to non-linear movement. Elastic deformation and hysteresis can further contribute to nonlinear stepping 701. Elastic deformation relates to temporary shape changes that occur under load and return to the original shape when the load is removed, whereas hysteresis relates to the lag between pulley rotation and instrument shaft articulation due to internal friction within the material of the shaft. During de-articulation, elastic components of the instrument shaft might not return to their original shape smoothly due to internal resistance, causing non-linear stepping. Uneven tension in the pull wires, or varying friction along the length of pull wire(s) can lead to inconsistent de-articulation as the forces are not evenly distributed, causing unpredictable steps. Wear and tear of instrument shaft and articulation system components can further introduce irregularities in movement, making the de-articulation process less predictable and less linear. For example, over time, mechanical components such as gears, pulleys, and wires can wear down, affecting their performance. Mechanical play and flexibility, such as relating to small movements between connected articulation system components and/or the degree to which the shaft and/or pull wire(s) can bend or flex under load, can further cause delays and irregular steps as the components settle into place during de-articulation. Furthermore, where the robotic control system is not configured to perfectly compensate for all mechanical imperfections, the articulation control algorithms or feedback mechanisms can lead to non-linear and unpredictable de-articulation movements.

The various articulation control issues demonstrated above relating to uncontrolled jumps/steps in instrument shaft articulation, particularly during de-articulation, can be mitigated through the introduction of articulation dampening, such as by implementing active and/or passive braking on articulation pull wire pulley(s). For example, with reference back to FIG. 4, the instrument base 11 can have associated therewith one or more articulation dampening brakes 64, which may be configured to apply friction to pull wire pulley(s) 33 to impede free rotation thereof. For example, the pulley(s) 33 can be coupled to or integrated with a robotic drive input 34, such as a gear receptacle or the like, configured to mate with a robotic drive. The pulley(s) 33 can fit or mate with the housing of the instrument base 11 in a manner as to allow for the pulley(s) 33 to rotate in a confined/secured position within the instrument base 11. Articulation dampening may be implemented by applying friction to the pulley(s) 33, thereby impeding rotation of the pulley(s) 33 and tensioning of the pull wire(s) 45.

The articulation dampening brake(s) 64 can be passive or active devices configured to transfer from the instrument base 11 to the pulley(s) 33, either directly on the pulley(s) 33 or to a structure coupled to or otherwise associated with the pulley(s) 33. For example, the brake(s) 64 can be any shape/form of one or more pads, blocks, discs, drums, clamps, calipers, shoes, stoppers, arrestors, stators, bands, or the like associated with the housing of the instrument base 11 and configured to resist rotation of the pulley(s) 33, either directly or indirectly, such as by surface contact between the brake and the pulley or pulley actuator. The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

The brake(s) 64 can be passive in terms of not being actively controllable, but rather being maintained in a chronic position within the base 11 in frictional contact with the pulley(s) 33. In some examples, the brake(s) 64 have the form of a drum brake against a shaft of a pulley or a disc brake against an axial side of the pulley wheel (e.g., a transverse face, surface, web portion) of a pulley. In some examples, the brake(s) 64 comprise a donut form positioned about a pulley shaft, each of the brake(s) is in contact with a pulley shaft and at least one surface of the instrument base 11.

In some implementations, the articulation dampening brake(s) 64 may be actively controllable, such that an amount of resistance to pulley rotation can change automatically or through active electronic and/or mechanical user control. For example, the brake(s) 64 may be electromechanically actuatable to press against a pulley 33, wherein the degree of pressure/friction and/or whether the brake contacts the pulley structure can be controlled by control circuitry of the robotic system.

Implementing articulation dampening as described herein can provide improved robotic instrument control, wherein an appropriate and/or optimal amount of friction force is determined and/or added to resist pulley rotation. FIG. 7B is a graph 700b showing a relationship between pulley rotation and pull wire tension for a partially plastic and elastic instrument shaft in accordance with one or more examples. For example, the graph 700b may correspond to the behavior of the instrument represented in the graph 700a of FIG. 7A. Generally, an effective amount of articulation dampening force for improving articulation control can correspond to an amount of pull wire load force experienced at the relevant pulley(s) that is associated with a point 711 at the end of (e.g., exiting) a direction change/reversal dead zone 705. Such an amount of added friction force can prevent a step-up in force when the pulley exits the dead zone 705 and encounters the tensioned resistance of the relevant pull wire.

The graph 700b shows example force levels 712 corresponding to dead zone boundaries 711 from the graph 700a. In some examples, the amount of force implemented for articulation dampening in accordance with the present disclosure is between 3-10 N, such as about 4 N, 4.5 N, 5 N, 5.5 N, 6 N, 6.5 N, 7 N, 7.5 N, 8 N, 8.5 N, 9 N, 9.5 N, 10 N, or other value. In some examples, about 7.5 N of frictional force is applied on a first pulley associated with a primary articulation plane and about 5 N of frictional force is applied on a second pulley associated with a secondary articulation plane. Once a desirable level of frictional force is determined and/or implemented, articulation of the instrument can be performed, wherein uncontrolled jumping/stepping in the instrument articulation may be mitigated due to the added articulation dampening force/friction.

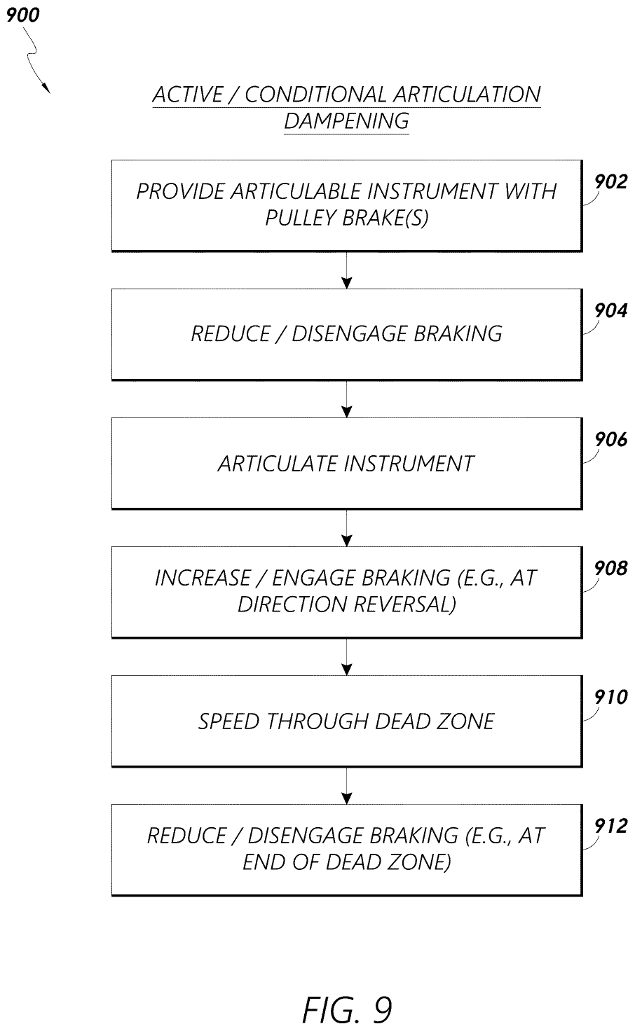

FIG. 8 is a graph 800 showing a relationship between pulley rotation and instrument deflection for a partially plastic and elastic instrument shaft implemented with articulation dampening in accordance with one or more examples. For example, the graph 800 may correspond to the instrument represented in the graph 700a of FIG. 7A, which is superimposed on the graph 800 for reference, wherein articulation dampening of the instrument has been implemented, such as with the use of a brake against a pull wire pulley, as described in detail herein.

With articulation dampening implemented, the instrument articulation response can advantageously be more linear, particularly during de-articulation. For example, unpredictable stepping during de-articulation of the instrument shaft, as represented by the marked de-articulation zones Dₐ, can advantageously be reduced in the graph 800 when compared to the graph 700a. Increases in de-articulation response linearity can be due to a decrease in backlash resulting from braking of the pull wire pulley(s) or other component(s) of the articulation system, which impedes jerky, uncontrolled movements in the pull wire pulley(s). The articulation dampening may further produce more linearity in the change-of-direction zones (i.e., ‘dead zones’) 803 due to the increased friction/resistance on articulation components resisting de-articulation until sufficient force has been applied to the pulley(s) to overcome the dampening friction/resistance. For example, such applied friction/resistance may be pre-determined and set to correspond to a threshold force associated with initiation of shaft de-articulation.

Although articulation dampening is disclosed herein primarily in the context of added friction on/against pull wire pulley component(s), in should be understood that articulation dampening in accordance with the present disclosure can involve applying friction/resistance to any suitable or desirable component of an articulation system. In some examples, articulation dampening may be implemented by increasing friction/tension on pull wires themselves. For example, friction can be added in an articulable segment of an instrument shaft, such as in the form of brakes, clamps, wedges, collars, or the like configured to apply friction directly on pull wire(s) within a distal articulable segment of the instrument shaft. However, implementation of articulation dampening within an articulable segment of an instrument shaft can lead to increased pull wire stretching due to the friction on the wires themselves, in addition to any stretching due to the natural elasticity of the instrument along the length of the shaft. Furthermore, added friction on the pull wires within the instrument shaft can produce higher force requirements through pull wire tubes in which the pull wire(s) are disposed (e.g., bowden tubes) and at attachment points between the pull wire(s) and the associated pulley(s), which can lead to increased instrument degradation. In addition, implementation of pull wire brakes within instrument shafts may be challenging due to the relatively small volumes within the distal articulable instrument shaft segment in which to dispose and/or operate such brake feature(s).

In some examples, articulation dampening may be implemented in a non-articulable segment of an instrument shaft. However, implementation of articulation dampening within a non-articulable segment of an instrument shaft can also lead to increased pull wire stretching due to the friction on the wires themselves, in addition to any stretching due to the natural elasticity of the instrument along the length of the shaft. Furthermore, added friction on the pull wires within the instrument shaft can produce higher force requirements through pull wire tubes in which the pull wire(s) are disposed and at attachment points between the pull wire(s) and the associated pulley(s), which can lead to increased instrument degradation. While the available area/volume within the non-articulable instrument shaft segment may be greater than that in the articulable segment, implementation of pull wire brakes in such segments may still be challenging due to the relatively small volumes. However, as the tubed (e.g., helixed coil pipes) in which the pull wires may be disposed in such segment can be relatively long traversing the non-articulable segment, greater surface area for contacting the pull wires and/or tubes may be available when compared to the area of the pull wires and/or tubes within the articulable segment. Therefore implementation of articulation dampening brake(s) within the non-articulable segment of an instrument shaft may be more feasible than within an articulable segment thereof. When implementing added friction on the pull wires themselves, articulation forces may increase due to opposing pull wires resisting one another, causing device degradation.

In some examples, articulation dampening may be achieved by reducing backlash between the robotic driver and the instrument base. However, such implementation can result in the loading and unloading of the instrument base from the robotic manipulator becoming more cumbersome and requiring tighter component tolerances, which can be relatively costly to manufacture. In some examples, articulation dampening may be achieved through the use of zero backlash drive system(s), such as magnetic drive systems. However, such systems can be associated with relatively high costs and/or added risk of slippage. In some examples, articulation dampening may be achieved by using two pulleys/motors per plane of articulation. Such implementations can enable robotic tensioning, such that at direction reversal, as directed by the system control circuitry, the tension need not need to be handed from one pull wire to the other across the reversal dead zone. However, such implementations require more motors to drive articulation, thus limiting the available degrees of freedom (e.g., fewer articulation sections, no shaft roll control, etc.).

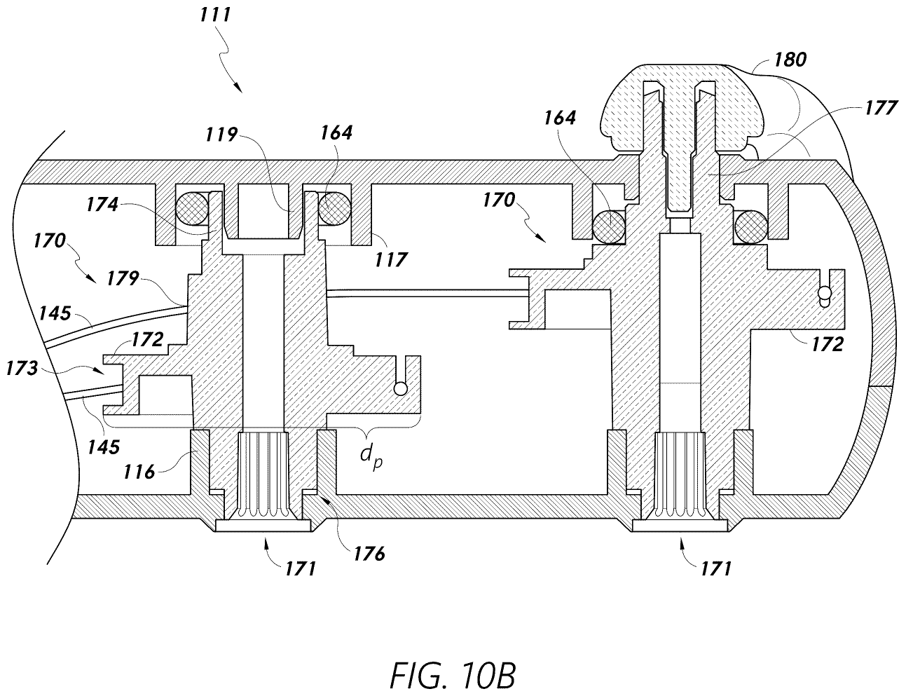

Compared to possible solutions described above in which articulation dampening brake(s) are implemented within a shaft of an instrument, implementation of articulation dampening brake(s) within an instrument base (e.g., handle) may be preferable for various reasons. For example, where braking is implemented to apply friction against a pulley within an instrument base (see, e.g., FIGS. 4, 10A, 10B), the amount of pull wire stretching may be less that of shaft-brake solutions, as no friction is applied directly to the pull wires, and therefore, pull wire stretching may result only from forces due to device elasticity. Pull wire and/or instrument degradation may likewise be less, as a pulley brake may advantageously not produce additional force through the pull wires. Furthermore, the instrument base may generally provide relatively greater space/volume for brake positioning and implementation, which can allow for implementation of more complex, effective, and/or accessible braking features.

In terms of how much friction is used to dampen articulation, such force can be approximated from the force and displacement plots of the instrument. In some implementations, for an articulable instrument with two planes of articulation (see, e.g., FIGS. 3, 4) approximately 7.5 N of friction may be added on a primary articulation plane/pulley and some lesser amount, such as approximately 5 N, may be added on the secondary plane/pulley to achieve desirable articulation response performance. Such degree of dampening may further produce bend-and-stay behavior in the instrument shaft, such that the shaft holds its shape once articulated after the articulation force is removed.

FIG. 9 is a flow diagram for a process 900 of controlling active articulation dampening in accordance with one or more embodiments. At block 902, the process 900 involves providing an articulable instrument including a base, a shaft, one or more pull wires disposed at least partially within the shaft and coupled to respective pulleys associated with the base. The instrument further includes a brake configured to apply friction to the articulation system of the instrument, such as a pulley brake. The pulley brake may be used to apply friction to the pull wire pulley(s) to improve articulation control, as described in detail herein. For example, in some implementations, an O-ring or other similar form or structure may be compressed and/or positioned between a pulley surface and a surface of the instrument base. The O-ring can be compressed to generate the desired amount of friction. There may be some risk that over time, components of the articulation system and/or the instrument will creep, thereby causing the amount of friction force applied by the brake to reduce. In addition, wear on the brake may occur through repeated use. Such changes can result in a loss of friction on the pulley and/or other component of the articulation system. Suitable material selection with respect to the pulley brake and/or other system components can help avoid the risks of component degradation or migration. In some implementations, relatively long spring features may be implemented in connection with the pulley brakes to provide consistent compression over a relatively long period.

In some implementations, the amount of compression (and friction) provided by a pulley brake may be adjusted in real time. At block 904, the process 900 involves reducing friction imposed by a pulley brake (e.g., disengaging the brake) prior to and/or during articulation of the instrument shaft. Alteration in brake force can be done using one or more electromagnets or using a motor. For example, in connection with the process 900, active braking may be implemented, wherein the amount of braking is adjusted as needed or engaged/released as needed. In some implementations, as indicated in block 906, the instrument may be articulated with the pulley brake(s) in a state of little or no friction applied. Such action can provide the relatively high sensitivity to allow for the use of force control to sense forces on, e.g., the tip of the instrument shaft.

At block 908, the process 900 involves engaging, or increase braking of the pulley brake(s). For example, when an articulation direction reversal is anticipated or determined, the process 900 may involve engaging or increasing braking of the pulley(s) to dampen instrument articulation. With the braking engaged/increased, the articulation drive motor may reverse with the shape of the instrument shaft held to some degree by the friction on the pull wire pulley(s). With the pulley(s) locked or restrained to some degree, the robotic system may be configured to drive the pulley(s) through the direction-reversal dead zone at an increase speed to reduce the user perception of the effect of the dead zone, thereby increasing apparent articulation control responsiveness. At block 910, the robotic drive may speed through the dead zone with the brake holding the articulation pulley(s) in place. When the robotic system detects a certain amount of force on one or more force/torque sensors of the articulation system (e.g., on the robotic drive output(s)), the force may be interpreted as the drive exiting the dead zone, which may cause the robotic system to slow the drive speed to match the controlled articulation speed. Articulation dampening in connection with the process 900 can be both during/after direction reversal (e.g., de-articulation) and during articulation. In some implementations, pulley friction may be added during de-articulation and removed during articulation.

At block 912, then amount of braking may be reduced, wherein the lowered level of friction force of the brake(s) may be maintained through the de-articulation stage as the instrument shaft returns towards a neutral position, possibly preceding further articulation of the instrument shaft in the reversal direction. Therefore, in connection with the process 900, articulation driving can be fully deterministic through both articulation and de-articulation. Furthermore, the control circuitry controlling articulation of the instrument shaft can be configured to account for any changes in the articulation response of the device throughout its life. The braking levels/parameters associated with the various stages of articulation control in the process 900 can be adjusted based on the determined control response of the instrument over time.

With reference back to FIG. 4, an instrument base 11 implemented with articulation dampening functionality in accordance with aspects of the present disclosure can include one or more pulley brakes 64 configured to apply continuous or selective friction to one or more pull wire tensioning pulleys or associated actuators. Such brake(s) 64 can take the form of a pad or ring that provides direct or indirect contact between the pulley/actuator and structure of the instrument base 11 (e.g., instrument base housing structure). In some implementations, the brake(s) 64 can comprise an O-ring disposed within and/or on the instrument base 11, wherein the O-ring is maintained in-place in contact with the pulley/actuator, such as within a groove, channel, chamber, or other volume/space. For example, the O-ring can wrap around a shaft 74 of a pulley, creating tension/dampening, as shown and described below in connection with FIGS. 10A and 10B.

FIGS. 10A and 10B show perspective and side cross-sectional views, respectively, of an instrument base 111 including one or more pulley brakes 164 in accordance with examples of the present disclosure. The instrument (e.g., endoscope) base 111 is coupled to an articulable shaft 140 that extends/projects distally from an outlet 193 of the base 111. The instrument base 111 can be configured to allow both manual shaft articulation control via a manual actuator 180 and robotic shaft articulation control via robotic drive input(s) 171. The manual actuator 180 can be configured as a lever, slider, wheel, or the like, which can be manually manipulated to provide manual two-way shaft articulation/deflection control. Moving the manual actuator 180 in a first direction can cause articulation of the elongate shaft 140 in a first articulation direction, and moving the manual actuator 180 in a second direction (opposite the first direction) can cause articulation of the elongate shaft in a second articulation direction in the same plane.

The instrument base 111 can include an electrical access 141 for power and/or data transfer. The instrument base 111 can include a working instrument inlet 143 that allows insertion of an instrument into a working channel of the shaft 140. The robotic drive input(s) 171 are configured to engage corresponding robotic drive output(s) when the instrument base 111 is attached to a robotic manipulator. The robotic drive output(s) (see, e.g., FIG. 3) can engage/mate with the drive input(s) 171 and transfer torque to rotate respective pulleys 170 associated with the drive input(s) 171. The pulleys 170 may be rotatable in both the clockwise and counterclockwise directions, as described herein. The robotic drive input(s) 171 can comprise grooved or keyed recesses and can be configured to engage robotic drive outputs that are configured as protruding splines, or vice versa. In some implementations, both a robotic drive input 171 and the manual actuator 180 can be configured to cause articulation of a common pulley 170.

In the illustrated example of FIGS. 10A and 10B, the instrument base 111 includes two pulley wheels 172, each part of, or otherwise associated with, a respective pulley 170. Each of the pulley wheels 172 can be associated with two of four articulation directions of the elongated shaft 140. The proximal end of the shaft 140 may have a bevel gear 149 associated therewith, wherein a drive gear is configured to mesh with the bevel gear 149 to cause rotation of the shaft 140 via rotation of the meshed drive gear. Pull wires 145 emanating from the pulley(s) 172 can run into the shaft 140. The pulley wheel(s) 172 can include one or more wire spool elements, wherein each pull wire spool element may be configured to spool a corresponding pull wire in a direction opposite the other. For example, a first pull wire spool element may spool a pull wire in a clockwise direction while a second pull wire spool element may spool a corresponding pull wire in a counterclockwise direction. Thus, each pulley wheel 172 can rotate in a first direction, causing one of the pull wires to unspool (e.g., extend) while causing another of the pull wires to spool (e.g., retract). In some embodiments, the instrument base 111 includes four pull wires, such that four-way deflection control is possible.

Each of the pulley(s) 170, an example implementation of one of which is described below individually for convenience, can include multiple axial segments, including a proximal segment that includes the robotic drive input gear receptacle 171. The proximal segment of the pulley 170 may fit within a bearing cup structure 116 of the instrument base 111. The cup structure 116 holds the pulley 170 in place, allowing it to rotate while remaining radially and axially constrained.

An intermediate/medial axial portion of the pulley 170 can include a pulley wheel 172, which may be positioned medially along a shaft or axle component/form 179 of the pulley 170. The pulley wheel 172 can project radially outward from the pulley shaft 179. The pulley wheel 172 can have a diameter dₚ more than twice that of the pulley shaft 179, providing suitable leverage for controlling the tension of the associated articulation control wire(s) 145. The pulley wheel 172 can include a circumferential groove 173, which may provide a channel in the radial periphery of the pulley wheel 172 that houses the associated pull wire(s) 145. The groove 173 can guide the pull wire(s) 145 in a manner as to facilitate smooth and controlled movement as the pulley wheel 172 rotates.

The pulley 170 can include a distal axial extension 174, 177 extending upwards (with respect to the orientation of FIG. 10B) from the pulley wheel 172, wherein the extension 174, 177 may include a retaining cap/cup that mates with a corresponding socket/projection structure 119 of the instrument base 111. The socket 119 can have the form of a pin, journal, stub axle, or similar component. Such arrangement can facilitate secure positioning of the pulley 170 while allowing for rotational movement thereof. The extension 174, 177, as well as the pulley itself 170, can be considered an axle component. A base collar 176 of the pulley 170 can fit into a dedicated bearing cup structure 116 (e.g., bushing) associated with a bottom surface/portion of the instrument base 111, wherein the bearing cup structure 116 can provide radial support and a rotational axis for the pulley 170, retaining the pulley 170 fixed in position while allowing rotational motion. The base collar 176 can include the input gear receptacle 171.

The instrument base 111 includes one or more articulation dampening brakes 164. In the example illustrated in FIGS. 10A and 10B, the articulation dampening brakes 164 are implemented as O-ring forms that are disposed between the axial extension 174, 177 (e.g., shaft) of the pulley 170 and a socket structure 117 or other fixed surface/structure of the instrument base 111. The O-ring brake 164 can provide indirect contact and friction between the pulley 170 and the housing of the instrument base 111. The O-ring brake 164 may comprise any suitable or desirable material. For example, the material of the O-ring brake may be selected to provide desirable characteristics with respect to wear resistance to withstand continuous friction without degrading quickly, friction coefficient to effectively increase resistance against the rotation of the pulley 170, chemical resistance with respect to any chemicals, oils, or lubricants that may be used in connection with the brake 164, and/or elasticity/flexibility to facilitate proper contact and pressure against the pulley extension 174, 177 and fixed socket 117. The material may further be selected to provide desirable characteristics with respect to compression set resistance to maintain effective sealing and friction over time, hardness/durometer that resists premature wearing-out and/or avoids damaging the pulley 170, and/or thermal stability with respect to the operating temperature range. In view of such considerations, rubber may be implemented for a material of the O-ring brake 164, such as nitrile rubber, fluorocarbon rubber, silicone rubber, ethylene propylene diene monomer (EPDM), neoprene/chloroprene rubber, or the like. In some implementations, the O-ring brake 164 comprises polyurethane, PTFE, or similar polymer. In particular, polyurethane, nitrile rubber, or fluorocarbon rubber may provide a desirable balance of wear resistance, friction, and thermal stability.

In some implementations, static friction associated with the O-ring brake 164 can be reduced through the addition of grease or other lubricant between the brake 164 and either or both of the pulley extension 174, 177 or the housing socket 117. For example, while the use of a lubricant may defeat a purpose of the brake 164 to some degree, which is designed to provide increased friction, whereas the lubricant would decrease the friction of the brake 164, lubricant may nevertheless be used to reduce static friction, provide smoother interfacing between the brake 164 and contacting components, and provide a particular predetermined/known amount of friction that can be accounted for by the system control circuitry.

The presence of one or more of the brake(s) 164 can provide dampening of articulation that results in more predictable, deterministic control with respect to pulley rotation and instrument shaft articulation response. In some implementations, the O-ring brake(s) 164 can provide sufficient friction to the pulley(s) 170 such that the instrument shaft becomes a bend-and-stay type shaft, wherein the articulation of the shaft is critically dampened. Furthermore, the brake(s) 164 can provide a known amount of friction, wherein the robotic system control circuitry can account for the known friction to provide articulation control that is perceived by the user as smooth, sensitive, and responsive. Implementation of articulation dampening brakes as passive devices like the O-rings 164 shown in FIGS. 10A and 10B can provide precise articulation control without the need for additional motors or the addition of excessive tension on the pull wires 145, which would reduce device life and/or consistency over the life of the system.

With reference back to FIG. 4, as described above, an instrument base 11 may have one or more rotation-actuated brakes 69 configured to adjust an amount of friction applied to a pull wire pulley based on a degree of rotation of the pulley. The brake(s) 69 can have any suitable or desirable form or mechanism configured to mechanically and/or electronically translate rotational position of a pulley to a corresponding friction state of the brake(s) 69. As an example, when the instrument shaft 40 is highly articulated, it may be desirable for an increased amount of friction to be applied by the brake(s) 69 to account for the higher likelihood of spring-back in such highly articulated states. Further, when the shaft 40 undergoes relatively little articulation, the brake(s) 69 may be configured to apply relatively less or no friction. The functionality of reducing pulley friction when the pulley moves to a rotational position associated with reduced articulation can be desirable as substantial friction on the pulley in such states can reduce the ability of the robotic system to determine the condition of the tip of the instrument shaft 40. In some implementations, rotation-actuated pulley braking may be implemented using a screw thread mechanism, which can translate pulley rotation into a braking contact pressure position of a brake in a manner as to set the amount of friction applied to the pulley to only the amount that is needed in each articulation state.

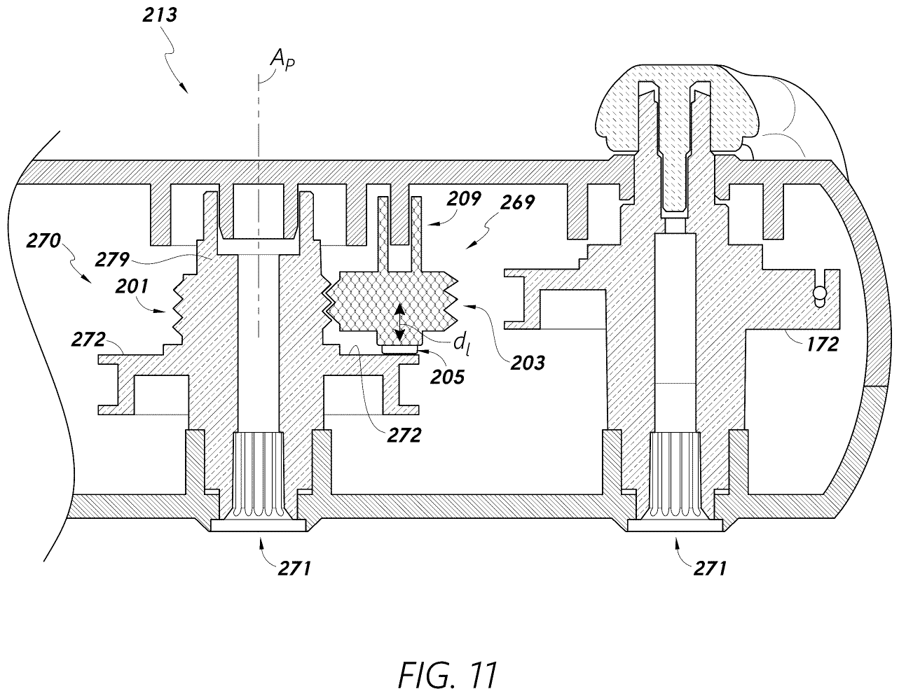

FIG. 11 shows an example mechanical implementation of a rotation-actuated brake 269 of an instrument base 213 in accordance with one or more examples. The base 213 includes a pulley 270 configured to rotate in a manner as to cause a change in tension of one or more pull wires coupled thereto, as described in detail herein. The brake 269 is implemented as a lead screw drive, wherein a shaft 279 of the pulley 270 operates as a lead screw, or power screw, that includes threads 201 designed to convert rotational motion of the shaft 279 to linear motion in the brake 269, which operates as a nut-type component that moves along an linear dimension dₗ that is parallel with an axis of the pulley 270. The mechanical configuration of the brake 29 and pulley 270 can advantageously provide a relatively simple and/or cost-effective mechanism for providing more consistent, precise control without the need to use additional motors or add excessive tension on the pull wires, which could reduce device life, consistency, and/or precision over time.

In operation, as the pulley 270 rotates about its axis Aₚ, the threads 201 of the pulley 270 push against the mated threads 203 of the brake 269, thereby translating the brake 269 in a direction along the dimension dₗ, namely the direction corresponding to the direction of rotation of the pulley 270. The brake 269 can include a brake pad 205, which is configured to press against the pulley wheel 272 and apply friction force thereto as the brake 269 moves in the downward direction (with respect to the illustrated orientation of FIG. 11) into contact with the pulley wheel 272. In some implementations, the brake pad 205 may be compressible, such that as the pad 205 is compressed, the amount of friction applied to the pulley wheel 272 increases through a range.

In some implementations, some rotational positions of the pulley 270 may cause the brake 269 to lift/stand off of the pulley wheel 272, such that no friction is applied to the pulley 270. For example, in some articulation states, it may be desirable to not implement dampening, whereas in other articulation states, a degree of dampening may be desirable. In some implementations, two brakes are used for a single pulley, wherein each brake is configured to apply friction to the pulley 270 in connection with one of the two articulation directions associated with the pulley 270. That is, when articulated in a first direction, a first one of the brakes may be disengaged from the pulley 270, such as by linear translation of the respective brake away from the pulley wheel 272 when the pulley 270 is rotated in a position associated with the first articulation direction, whereas a second one of the brakes may be configured to apply friction in at least some of the positions associated with the first articulation direction. When the pulley is rotated to a position associated with articulation of the instrument in a second direction, the second brake may be disengaged from the pulley, whereas the first brake may be configured to apply friction to the pulley 270 in at least some of the positions associated with the second articulation direction.

The brake 269 can be secured/supported by a bearing structure 209 configured to provide a low-friction interface for rotation of the brake 269, while also supporting/confining the position of the brake 269 within the base 213. The drive input 271 serves as a coupler that couples the pulley 270 to a robotic drive output for transmission of rotational force to the pulley 270.

### Additional Description of Examples and Terminology

Provided below is a list of examples, each of which may include aspects of any of the other examples disclosed herein. Furthermore, aspects of any example described above may be implemented in any of the numbered examples provided below.

Example 1: An instrument comprising a base, a shaft that projects from the base and has one or more articulation pull wires disposed at least partially therein, a robotic drive input, a pulley having at least one of the one or more articulation pull wires coupled thereto, and a pulley brake configured to apply friction to the pulley to provide resistance to rotational movement of the pulley, wherein the pulley is rotatable about an axis by the robotic drive input.

Example 2: The instrument of any example herein, in particular example 1, wherein the pulley brake comprises a compressible form disposed in physical contact with the pulley and a housing of the base.

Example 3: The instrument of any example herein, in particular example 1, wherein the pulley brake comprises an O-ring disposed about a shaft of the pulley.

Example 4: The instrument of any example herein, in particular example 3, wherein the shaft is positioned on a first axial side of a pulley wheel of the pulley and the robotic drive input is positioned on a second axial side of the pulley wheel.

Example 5: The instrument of any example herein, in particular example 3, wherein the O-ring is disposed between an axle of the pulley and a bearing housing of the base.

Example 6: The instrument of any example herein, in particular example 5, wherein the O-ring is in compressed contact with both the axle and the bearing housing.

Example 7: The instrument of any example herein, in particular example 6, further comprising a lubricant on a surface of the O-ring that reduces static friction of the O-ring.

Example 8: The instrument of any example herein, in particular example 1, wherein the pulley brake is controllable to adjust an amount of friction force applied to the pulley.

Example 9: The instrument of any example herein, in particular example 1, wherein the pulley brake is configured to apply the friction directly to an axial shaft of the pulley.

Example 10: The instrument of any example herein, in particular example 1, wherein the pulley brake is configured to apply the friction directly to a transverse face of a pulley wheel of the pulley.

Example 11: The instrument of any example herein, in particular example 1, wherein the pulley brake is configured to adjust an amount of friction applied to the pulley based on a rotational position of the pulley.

Example 12: The instrument of any example herein, in particular example 11, wherein the pulley brake is in threaded engagement with the pulley, such that rotation of the pulley actuates the pulley brake.

Example 13: The instrument of any example herein, in particular example 11, wherein rotation of the pulley causes axial linear translation of the pulley brake.

Example 14: An instrument comprising an elongate shaft having associated therewith a plurality of articulation pull wires that run a length of the elongate shaft, one or more robotically actuatable pulleys configured to tension the plurality of articulation pull wires, pulley braking means configured to apply friction to a first pulley of the one or more robotically actuatable pulleys.

Example 15: The system of any example herein, in particular example 14, wherein the pulley braking means comprises a shoe brake configured to apply friction against a rotating shaft of the first pulley.

Example 16: The system of any example herein, in particular example 14, wherein the pulley braking means comprises a disc brake configured to apply friction against a transverse face of a pulley wheel of the first pulley.

Example 17: The system of any example herein, in particular example 14, wherein the pulley braking means is controllable to adjust an amount of friction applied to the first pulley.

Example 18: A robotic system comprising a robotic end effector comprising one or more drive outputs configured to cause articulation of an elongate shaft of an instrument coupled to the robotic end effector at least in part by causing a pulley of the instrument to rotate, and control circuitry configured to determine a rotation position of the pulley and cause braking to be applied to the pulley based at least in part on the rotation position.

Example 19: The robotic system of any example herein, in particular example 18, wherein the control circuity is further configured to, in response to a determination that the rotation position is associated with an articulation dead zone, cause an amount of braking applied to the pulley to be increased and cause a speed of rotation of the pulley to be increased.

Example 20: The robotic system of any example herein, in particular example 18, wherein the control circuitry is further configured to cause an amount of braking applied to the pulley to be increased in response to a determination that the rotation position is associated with a high degree of articulation of the elongate shaft of the instrument.

For purposes of summarizing the disclosure, certain aspects, advantages, and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An instrument comprising:
a base;
a shaft that projects from the base, the shaft having one or more articulation pull wires disposed at least partially therein;
a robotic drive input;
a pulley having at least one of the one or more articulation pull wires coupled thereto, the pulley being rotatable about an axis by the robotic drive input; and
a pulley brake configured to apply friction to the pulley to provide resistance to rotational movement of the pulley.

2. The instrument of claim 1, wherein the pulley brake comprises a compressible form disposed in physical contact with the pulley and a housing of the base.

3. The instrument of claim 1, wherein the pulley brake comprises an O-ring disposed about a shaft of the pulley.

4. The instrument of claim 3, wherein:
the shaft is positioned on a first axial side of a pulley wheel of the pulley; and
the robotic drive input is positioned on a second axial side of the pulley wheel.

5. The instrument of claim 3, wherein the O-ring is disposed between an axle of the pulley and a bearing housing of the base.

6. The instrument of claim 5, wherein the O-ring is in compressed contact with both the axle and the bearing housing.

7. The instrument of claim 6, further comprising a lubricant on a surface of the O-ring that reduces static friction of the O-ring.

8. The instrument of claim 1, wherein the pulley brake is controllable to adjust an amount of friction force applied to the pulley.

9. The instrument of claim 1, wherein the pulley brake is configured to apply the friction directly to an axial shaft of the pulley.

10. The instrument of claim 1, wherein the pulley brake is configured to apply the friction directly to a transverse face of a pulley wheel of the pulley.

11. The instrument of claim 1, wherein the pulley brake is configured to adjust an amount of friction applied to the pulley based on a rotational position of the pulley.

12. The instrument of claim 11, wherein the pulley brake is in threaded engagement with the pulley, such that rotation of the pulley actuates the pulley brake.

13. The instrument of claim 11, wherein rotation of the pulley causes axial linear translation of the pulley brake.

14. An instrument comprising:
an elongate shaft having associated therewith a plurality of articulation pull wires that run a length of the elongate shaft;
one or more robotically actuatable pulleys configured to tension the plurality of articulation pull wires; and
pulley braking means configured to apply friction to a first pulley of the one or more robotically actuatable pulleys.

15. The instrument of claim 14, wherein the pulley braking means comprises a shoe brake configured to apply friction against a rotating shaft of the first pulley.

16. The instrument of claim 14, wherein the pulley braking means comprises a disc brake configured to apply friction against a transverse face of a pulley wheel of the first pulley.

17. The instrument of claim 14, wherein the pulley braking means is controllable to adjust an amount of friction applied to the first pulley.

18. A robotic system comprising:
a robotic end effector comprising one or more drive outputs configured to cause articulation of an elongate shaft of an instrument coupled to the robotic end effector at least in part by causing a pulley of the instrument to rotate; and
control circuitry configured to:
determine a rotation position of the pulley; and
cause braking to be applied to the pulley based at least in part on the rotation position.

19. The robotic system of claim 18, wherein the control circuitry is further configured to:
in response to a determination that the rotation position is associated with an articulation dead zone:
cause an amount of braking applied to the pulley to be increased; and
cause a speed of rotation of the pulley to be increased.

20. The robotic system of claim 18, wherein the control circuitry is further configured to cause an amount of braking applied to the pulley to be increased in response to a determination that the rotation position is associated with a high degree of articulation of the elongate shaft of the instrument.
